(19) United States
(12) Patent Application Publication
(10) Pub. No.: US 2010/0179625 A1
(43) Pub. Date: Jul. 15, 2010

(54) Implantable Heating Apparatus for a Living Being and Method for Charging the Same

(75) Inventors: Fu Jen Kao, Taipei (TW); Cheng Chun Li, Taipei (TW); Cheng Chi Wu, Taipei (TW)

Correspondence Address: Hogan Lovells US LLP, 1999 AVENUE OF THE STARS, SUITE 1400, LOS ANGELES, CA, 90067, US

(73) Assignee: National Yang-Ming University, Taipei (TW)

(21) Appl. No.: 12/432,096

(22) Filed: Apr. 29, 2009

(30) Foreign Application Priority Data
Jan. 13, 2009 (CN) 098101014

(51) Int. Cl. A61F 7/12

(52) U.S. Cl. 607/113

(57) ABSTRACT

An implantable heating apparatus for a living being of the present invention includes a heating unit, a control unit for controlling operations of the heating unit and an induction driven charge/discharge unit for powering the heating unit. The induction driven charge/discharge unit is composed of a core, a coil set wrapping around the core in at least three axial directions and an energy storing unit for electrically coupling to the coil set. Such that when an external alternate magnetic field is approaching the induction driven charge/discharge unit, the coil set is able to generate induction current that can be stored in the energy storing unit.

### CROSS REFERENCE

Not applicable

### BACKGROUND OF THE INVENTION

1. Field of the Invention

The present invention is related to a heating apparatus, and more particularly, to an implantable heating apparatus for a living being capable of storing energy therein and the method for charging the heating apparatus.

2. Description of Related Art

Heat treatment can be traced back to 5000 BC and the German was enthusiastic at the treatment in the early 19 century. Back in the early days, the heat treatment was relatively primitive. During the times, some doctors even used heated needle or burned iron to engage lumps of a patient directly. Recently, numerous countries have conducted various tumor heat treatment studies as auxiliary means for treating cancer patients during the mid-to-late periods. The heat treatment has been increasingly applied clinically and presents to be one of the most effective treatments used for tumor patients after operations, radioactive treatment, chemical therapy and bio-target therapy. Each day, one gram of tumor tissue can release millions of cancer cells to blood running through all parts of the human body and one per million of the cancer cells may potentially survive. When the cancer cells are trying to transfer or grow into metastatic cancer, the cancer cells need to develop new vessels. Heat treatment is effective in inhibiting the growth factor of a new blood vessel and thus blocks the cancer cells' metabolism and damage the genetic substance thereof. Subsequently, the cancer cells stop proliferating so that the cancer reoccurrence or transference is inhibited. Additionally, the heat treatment will result in incomplete growth of blood vessels inside the tumor and the temperature rise will increase the resistance of the blood vessels of the tumor and the viscosity of the blood, which induces the formation of thrombus and thus inhibits the formation of tumors. The cancer cells are, therefore, destroyed.

In related arts, a magnetic material functioning as a heating device is implanted inside an animal to generate internal heating. By means of the generation of an alternate magnetic field by the magnetic field emitting unit, the heating device is able to form an induction current (eddy current) under the influence of the alternate magnetic field, which generates heat to raise temperature surrounding the specified part of the animal. U.S. Pat. No. 6,397,107 is related to the use of an external high frequency power source in connection to an induction coil; such that a metal coil implanted inside the human body and functioned as a magnetic field receiving device is able to generate induction current. Heat generated from the induction current causes the blood vessels to mutate, for example, aneurysm and condensation or contraction happens. These two related arts deal with the induced heating device operated and controlled by an external magnetic field. None of which is equipped with any coping device at the point where the induction current is generated when the orientation of the magnetic field is changed. So that the induction current generating efficiency is greatly reduced. Also, the control of the heating device is purely dependent on the external magnetic field and there is no way to meet the specific requirements, such as the adjustment of temperature, the heating rate, etc.

As a result of the deficiencies caused by conventional arts, it is crucial to consider how to cope with the orientation change of the magnetic field for the induced coil being able to effectively generate induced current to charge the heating device.

### SUMMARY OF THE INVENTION

An object of the present invention is to provide an implantable heating apparatus for enabling superior energy management and efficiency.

Another object of the present invention is to provide an implantable heating apparatus for achieving multiple heating modalities.

In order to describe the invention in detail, it is provided that the implantable heating apparatus for a living being of the present invention includes, but not limited to, a heating unit, a control unit for controlling operations of the heating unit and an induction driven charge/discharge unit for powering the heating unit. The induction driven charge/discharge unit is composed of a core, a coil set wrapping around the core in at least three axial directions and an energy storing unit for electrically coupling to the coil set. Such that when an external alternate magnetic field is approaching the induction driven charge/discharge unit, the coil set is able to generate induced current and the induced current is then stored in the energy storing unit.

A further object of the present invention is to provide an energy storing method for an implantable heating apparatus for a living being. The method includes the steps of: providing a coil set for wrapping around a core in at least three axial directions, electrically coupling the coil set, an energy storing unit, a heating unit and a controlling unit for controlling operations of the heating unit and providing an alternate magnetic field for generating induced current to be stored in the energy storing unit.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles as well as the best mode of the invention.

FIG. 1 is a schematic view showing the preferred location of the heating apparatus of the present invention;

FIG. 2 is a block diagram of the heating apparatus of the present invention;

FIG. 3 is a partial plan view showing an core with a coil set used in the heating apparatus of the present invention;

FIGS. 4A to 4G are perspective views showing the embodiments of the core of the present invention; and

FIG. 5 is a flowchart showing the process of operating the heating apparatus of the present invention.

### DESCRIPTION OF EMBODIMENTS

Faraday principle describes: the magnitude of the electromagnetic force (EMF) in a circuit is equal to the variation of the magnetic flux in a given period of time. And the direction of the induced EMF is in the direction opposite to that of the magnetic flux variation.

The preferred embodiment of the present invention is designed based on the Faraday principle and is associated with an alternate magnetic field to generate a magnetic field so that under the influence of induction, a coil set is able to generate an alternate current to be stored in an energy storing unit.

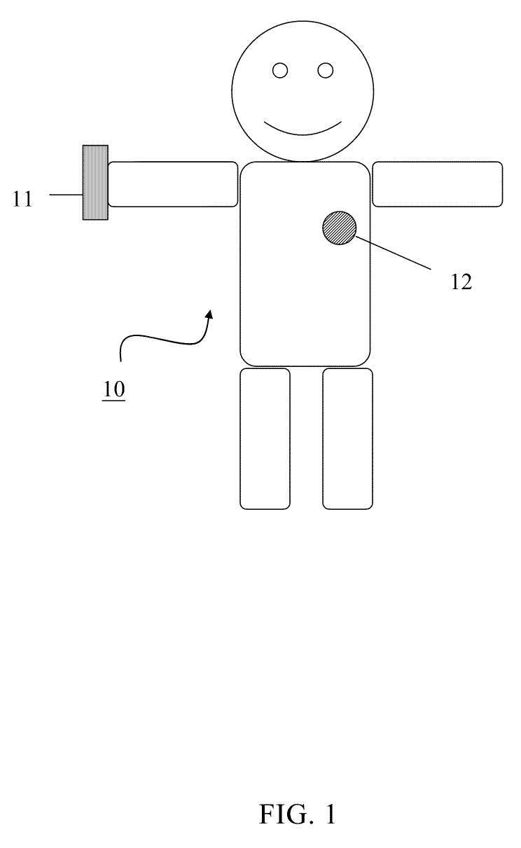

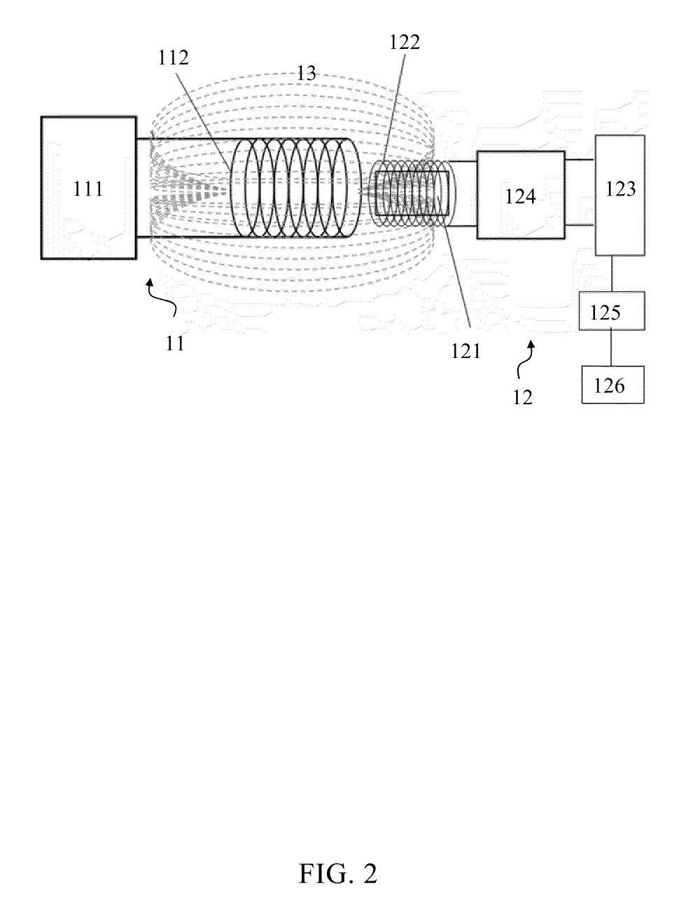

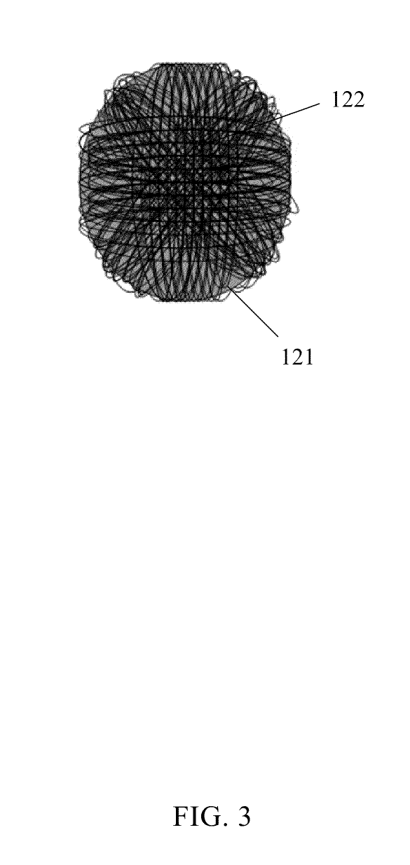

Referring to FIGS. 1, 2 and 3, it is noted that the preferred embodiment of the present invention is shown in each of the drawings and the implantable heating apparatus 12 is composed of a heating unit 126, a controlling unit 125 for controlling operations of the heating unit 126 and an induction driven charge/discharge unit for powering the heating unit 125. The induction driven charge/discharge unit includes a core 121, a coil set for wrapping around the core 121 in at least three axial directions and an energy storing unit 123 for electrically coupling to the coil set 122. When an external alternate magnetic field approaches the heating apparatus 12 of the preferred embodiment of the present invention, the coil set 122 is able to generate induced current to be stored in the energy storing unit 123. The external alternate magnetic field 13 is generated by a magnetic field generator 11 which is composed of an operating unit 111 and a high frequency coil 112 coupled with the operating unit 111. Therefore, a user may use the operating unit 111 to generate an alternate magnetic field 13 around the high frequency coil 112. That is, the operating unit 111 varies the current strength on the high frequency coil 112 to be dependent on time; consequently the strength of the magnetic field is controlled. It is noted that a current detector (not shown) may be coupled between the operating unit 111 and the high frequency coil 112 to avoid the high frequency coil 112 from being damaged by a sudden surge of the electrical current. In addition, the heating unit 126 is a resistance heater.

When the alternate magnetic field 13 is approaching the heating apparatus 12, the coil set 122 wrapping around the core 121 responds to the alternate magnetic field 13 and generates electrical current, which, in turn is stored in the energy storing unit 123. Since the coil set 122 is wrapped around the core 121 in at least three axial directions, such as X, Y, and Z axes (vertical to each other), any direction change of the alternate magnetic field will have an angle approximate less than 45 degree with at least one axes of the coil set 122, which greatly increases the induced current efficiency. Besides, the coil set 122 may be wrapped around the core 121 in 45 degrees between any two axes of X, Y, and Z axes of the core 121 to further increase the efficiency of the generation of the induced current. The coil set 122 may contain only one cable to wrap around the core 121 or may contain plural cables respectively wrapping around the core 121 in the previously designed axial directions and are electrically coupled to the energy storing unit 123 in parallel for providing the induced current to the energy storing unit 123 independently. For example, the coil set contains one cable for wrapping around the coil in different directions. Moreover, the coil set 122 is wrapped around the core 122 in three different axial directions, i.e., X, Y and Z, the coil set 122 contains three different cables; or the coil set 122 contains six (6) different cables if the coil set 122 is wrapped around the core 122 in 45 degrees relative to the three different axes of the core 121. Furthermore, a rectifier 124 may be coupled between the coil set 122 and the energy storing unit 123 to protect the energy storing unit 123 from damage by a sudden surge of the induced current. The energy storing unit 123 may be a rechargeable battery made by any method and material known in the art.

After placing the heating apparatus 12 of the preferred embodiment of the present invention present invention inside a human body (i.e., closed to ribs of the human body), the user or medical personnel may remotely control the controlling unit 125 to activate the heating unit 126 and to control or adjust the heating period, heating temperature and heating areas to fully satisfy the requirements under all kinds of situations. For example, the medical personnel may control the heating unit 126 to heat instantly so as to provide the necessary heating effect to a particular area of the human body or the medical personnel may control the heating unit 126 to heat up slowly for a prolonged period of time so as to increase the flexibility as well as the effects of the healing process. When the heating period is prolonged, the energy in the energy storing unit 123 may gradually run out. To charge the energy of the energy storing unit 123, the user may place the magnetic field generator 11 at a place close to where the heating apparatus 12 is situated, the alternate magnetic field 13 generated by the magnetic field generator 11 can charge the energy storing unit 123. During the charging process, if the magnetic field generator 11 shifts its location due to movement of the user, the direction of the alternate magnetic field 13 also changes. However, because the coil set 122 is wrapped around the core in more than one single direction relative to the core, regardless of the directional change of the alternate magnetic field 13, the magnetic field generator 11 may still generate effective induced current to charge the energy storing unit 123 within a reasonable distance range. Still further, the controlling unit 125 contains a temperature sensor (not shown) for sensing temperature change of the surroundings of the heating unit 126. Also, the changed temperature information may be fed back to control the heating unit 126 for maintaining the heating temperature of the heating unit 126 within a reasonable range to proceed the heating treatment.

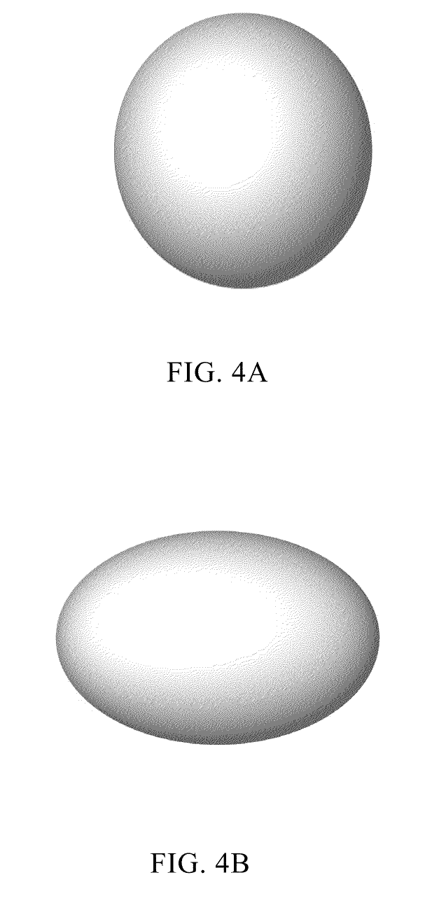

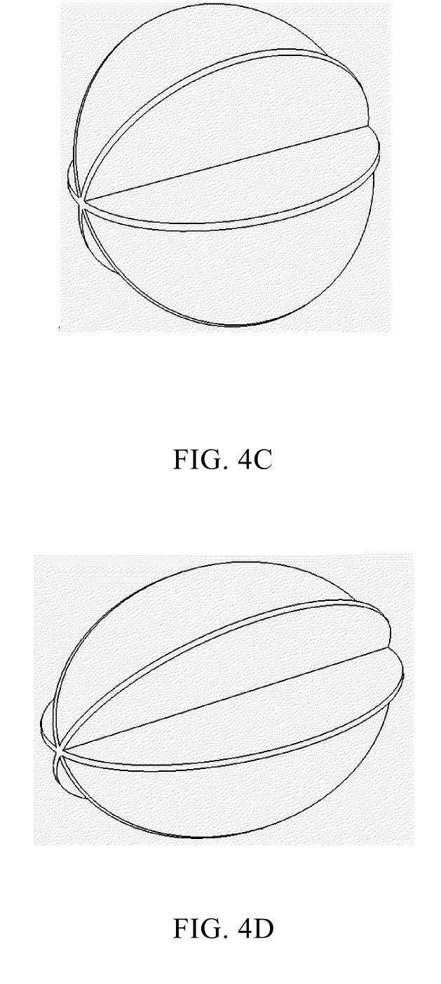

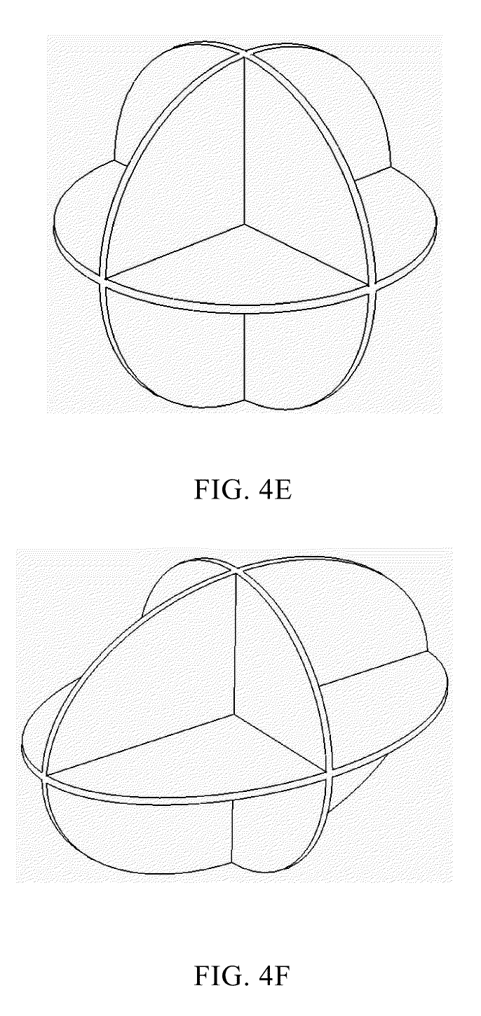

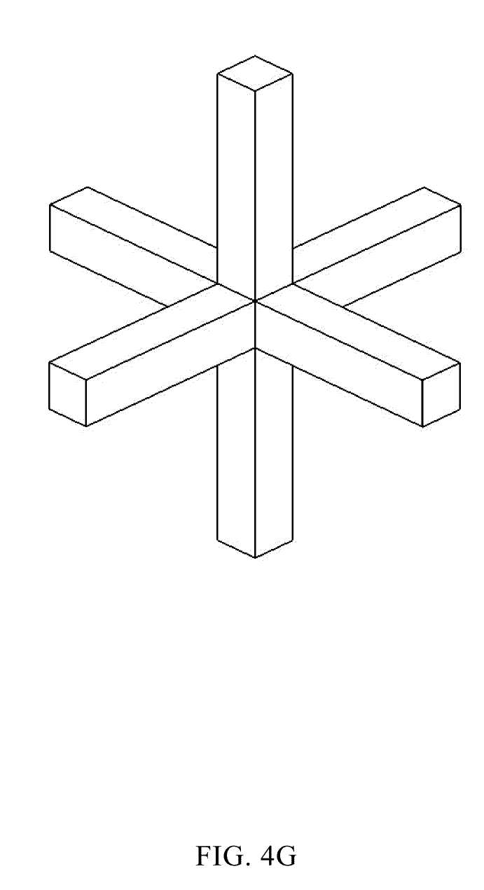

Referring to FIGS. 4A to 4G, it is noted that the core 121 may be a solid body or a hollow body and may be shaped as a sphere, a cylinder, an elliptical sphere, a body made of three cylinder or elliptical plates interactively crossed with one another in an ordered manner, so that the cables from the coil set may be wrapped around to accomplish the previously designed objects. Moreover, the core 121 is made of a soft magnetic material such as cobalt or nickel.

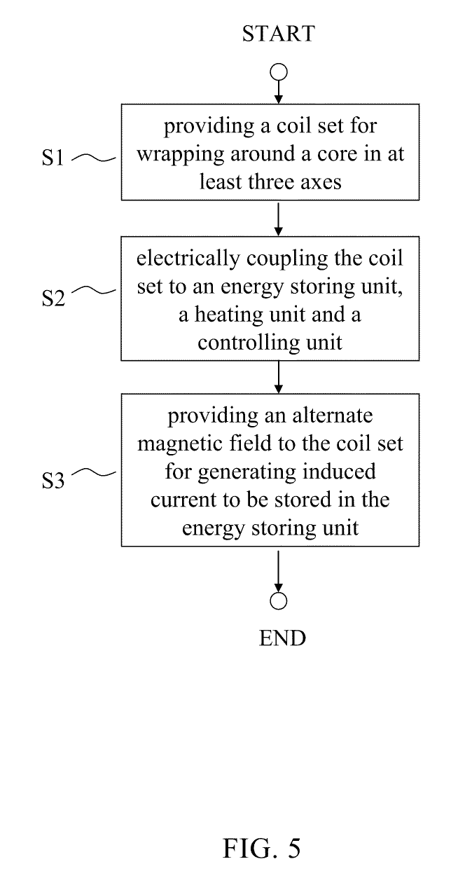

Referring to FIG. 5, it is noted that the method for charging the energy storing unit comprises the steps of: providing a coil set for wrapping around a core in at least three axes (X, Y, Z, the axial directions from this embodiment may be increased up to six (6) axes) (S1); electrically coupling the coil set to an energy storing unit, a heating unit and a controlling unit (S2); and providing an alternate magnetic field to the coil set for generating induced current to be stored in the energy storing unit (S3) to complete the charging process.

In addition, before initiating S1, the core may be a sphere, elliptical sphere or a column made of three respective circular plates interactively crossed with one another so as to provide easy access to the cables when wrapping around the core. The core may be made of a soft magnetic material such as soft iron, cobalt or nickel so as to increase the generation of the induced current. Before step S2 is executed, a rectifier may be coupled between the coil set and the energy storing unit so as to protect the energy storing unit from damage by a sudden current surge. Moreover, the energy storing unit may be a rechargeable battery and made by any know method in the art.

Furthermore, the alternate magnetic field is generated by a magnetic field generator which is composed of an operating unit and a high frequency coil so that the user may use the high frequency coil to generate an alternate magnetic field by the function of the operating unit. That is, the user may use the operating unit to control the strength of the magnetic field. A current sensor may be coupled between the operating unit and the high frequency coil to protect the high frequency coil from damage by a sudden current surge.

The heating apparatus of the preferred embodiment of the present invention uses cables to wrap the hollow spherical core completely such that there will be no blind spot for induction of the magnetic field. As a matter of this design, limitations such as lack of space and inefficient charging to the energy storing unit are overcome.

Compared with the existing heating devices currently used in the market, it is notable that because the wrapping method of the cable of the preferred embodiment, there is almost no directional limitation in the induction of the current, so that the efficiency in generating induced current is greatly improved. Furthermore, the stored energy is easily transformed into heat for treating. The user may adjust the heating process mode such as the heating time period, the heating speed and the heating area for allowing the heat treatment being much more flexible to satisfy various requirements. The heating apparatus of the preferred embodiment may be stayed inside the human body for a prolonged period of time without harming the human health.

Although the present invention has been disclosed above by preferred embodiments, they are not intended to limit the present invention. Anybody skilled in the art can make some modifications and alterations without departing from the spirit and scope of the present invention. Therefore, the protecting range of the present invention falls in the appended claims.

What is claimed is:

1. An implantable heating apparatus for a living comprising:
a heating unit;
a controlling unit coupled to the heating unit for controlling the operations of the heating unit; and
an induction driven charge/discharge unit for powering the heating unit, the induction driven charge/discharge unit including:
a core;
a coil set for wrapping around the core in at least three axial directions; and
an energy storing unit for electrically coupling to the coil set;
wherein when an external alternate magnetic field approaches the induction driven charge/discharge unit, the coil set is able to generate induced current to be stored in the energy storing unit.

2. The implantable heating apparatus as claimed in claim 1, wherein the alternate magnetic field is generated by a magnetic field generator and the magnetic field generator is composed of an operating unit and a high frequency coil coupled with the operating unit with which electrical current of the high frequency coil is able to be adjusted dependently on time.

3. The implantable heating apparatus as claimed in claim 1, wherein the core is wrapped around by the coil set in at least three axial directions vertical to each other.

4. The implantable heating apparatus as claimed in claim 1, wherein the core is wrapped around by the coil set in X, Y and Z axes of the core.

5. The implantable heating apparatus as claimed in claim 4, wherein the core is further wrapped around by the coil set in 45 degrees relative to any two axes of the X, Y and Z axes.

6. The implantable heating apparatus as claimed in claim 1, wherein the coil set contains a single cable to wrap around the core.

7. The implantable heating apparatus as claimed in claim 1, wherein the coil set contains a plurality of cables respectively wrapping around the core and in parallel with respect to the energy storing unit.

8. The implantable heating apparatus as claimed in claim 1, wherein a rectifier is coupled between the coil set and the energy storing unit.

9. The implantable heating apparatus as claimed in claim 1, wherein the energy storing unit is a rechargeable battery.

10. The implantable heating apparatus as claimed in claim 1, wherein the heating unit is a resistance heater.

11. The implantable heating apparatus as claimed in claim 1, wherein the core is selected from the group consisting of a sphere, an elliptical sphere and a cylinder.

12. The implantable heating apparatus as claimed in claim 1, wherein the core is composed of three cylinders, circular plates or elliptical plates interactively crossed with one another.

13. The implantable heating apparatus as claimed in claim 1, wherein the core is made of a material selected from the group consisting of soft magnet, cobalt or nickel.

14. The implantable heating apparatus as claimed in claim 1, wherein the core may be hollow or solid.

15. The implantable heating apparatus as claimed in claim 1, wherein the controlling unit is able to control heating time, temperature, heating speed and heating area of the heating unit.

16. The implantable heating apparatus as claimed in claim 1, wherein the controlling unit further comprises a temperature sensor for detection of temperature change of and around the heating unit as an auxiliary control over the heating unit for heating.

17. An energy storing method for an implantable heating apparatus for a living being comprising the steps of:
providing a coil set for wrapping around a core in at least three axial directions;
electrically coupling the coil set to an energy storing unit, a heating unit and a controlling unit responsible for controlling the heating unit; and
providing an alternate magnetic field for allowing the coil set to generate induced current to be stored in the energy storing unit.

18. The method as claimed in claim 17, wherein the core is further wrapped around by the coil set in 45 degrees relative to any two axes of the X, Y and Z axes.

19. The method as claimed in claim 17, wherein the coil set contains a single cable responsible for wrapping around the core.

20. The method as claimed in claim 17, wherein coil set contains a plurality of cables respectively wrapping around the core and in parallel with respect to the energy storing unit.

21. The method as claimed in claim 17 further comprising a rectifier for coupling between the coil set and the energy storing unit.

22. The method as claimed in claim 17, wherein the core is made of a material selected from the group consisting of soft magnet, cobalt or nickel.

23. The method as claimed in claim 17, wherein the alternate magnetic field is generated by a magnetic field generator which is composed of an operating unit and a high frequency coil coupled to the operating unit, the controlling unit being able to vary the current strength on the high frequency coil to be dependent on time

24. The method as claimed in claim 23, wherein a current detector is coupled between the operating unit and the high frequency coil for protecting the energy storing unit from damage of a sudden current surge.
